(19) United States
(12) Patent Application Publication
(10) Pub. No.: US 2015/0325036 A1
(43) Pub. Date: Nov. 12, 2015

(54) APPARATUS AND METHOD FOR SCAN IMAGE DISCERNMENT IN THREE-DIMENSIONAL ULTRASOUND DIAGNOSTIC APPARATUS

(71) Applicant: KOREA DIGITAL HOSPITAL EXPORT AGENCY, Seoul (KR)

(72) Inventor: Min Hwa LEE, Seoul (KR)

(21) Appl. No.: 14/423,709

(22) PCT Filed: Aug. 27, 2013
(86) PCT No.: PCT/KR2013/007661

§ 371 Date: Jul. 20, 2015

(30) Foreign Application Priority Data
Aug. 27, 2012 (KR) 10-2012-0093717

(51) Int. Cl. G06T 15/08; G06T 5/00; A61B 5/11; G06T 15/40; A61B 8/08; A61B 8/00

(52) U.S. Cl. CPC G06T 15/08; A61B 8/483; A61B 8/4254; A61B 8/461; A61B 8/5215; A61B 8/5276; A61B 5/11; G06T 15/40; G06T 5/002; G06T 2207/10132

(57) ABSTRACT

Disclosed is an apparatus of determining a scan image of a three-dimensional ultrasound diagnostic apparatus, including: a scanning unit configured to generate two-dimensional volume images of an inside of a human body using an ultrasonic signal; a processing unit configured to combine the two-dimensional volume images acquired through the scanning unit to generate a three-dimensional volume image and determine whether the three-dimensional volume image is normal; a database configured to store the three-dimensional volume image generated by the processing unit; and an alarm sound output unit configured to, when data determined by the processing unit is not normal, provide notice thereof using any one or more of an alarm sound, an alarm light, and an alarm message.

### TECHNICAL FIELD

The present invention relates to an apparatus and method for scan image discernment in a three-dimensional ultrasound diagnostic apparatus, and more particularly, to an apparatus and method for discerning an inaccurate or incorrect scan image and providing notice thereof using an alarm sound, an alarm light, and/or an alarm message in a three-dimensional ultrasound diagnostic apparatus.

### BACKGROUND ART

In general, an ultrasonic image diagnostic apparatus, which emits an ultrasonic wave to a human body, detects a reflection wave returned from the human body, performs appropriate signal processing, and displays the processing result on a screen, has been widely used in a medical field since an image of an internal organ may be observed in real time without needing to make an incision in the body.

The ultrasonic image diagnostic apparatus tends to switch from analog to digital and switch from a two-dimensional ultrasound diagnostic apparatus to a three-dimensional or four-dimensional ultrasound diagnostic apparatus, and provides a stereoscopic image as a real-time video and facilitates remote diagnosis from a remote place through a volume image network.

For the remote diagnosis, three-dimensional data obtained through field inspection is transmitted to a large hospital or a hospital having a medical team who can diagnose an ultrasonic image. In this case, if an image of the transmitted data is not clear, a photograph should be retaken with additional time and cost.

### DISCLOSURE

### Technical Problem

The present invention is directed to determining whether an image is shaken or has a hidden part through a blurring determination algorithm and an image check algorithm when scanning an organ of a patient, and instructing rephotographing to transmit a clear three-dimensional volume image to a screening place when the scanning is not correct.

The present invention is also directed to determining whether there is an error through a blurring determination algorithm and a hiding determination algorithm of a three-dimensional ultrasound diagnostic apparatus and a three-axis acceleration sensor of a three-dimensional ultrasonic probing unit, and correcting the error.

### Technical Solution

One aspect of the present invention provides a method including a first step of determining a measurement position of a probing unit on a human body, a second step of determining a measurement angle of the probing unit, a third step of detecting a tremor of a hand holding the probing unit or shaking of the human body, a fourth step of detecting noise or blurring phenomena in a three-dimensional volume image, and a fifth step of determining whether organs intended to be measured have all been scanned.

Another aspect of the present invention provides an apparatus including a scanning unit configured to generate two-dimensional volume images of an inside of a human body using an ultrasonic signal, a processing unit configured to combine the two-dimensional volume images acquired through the scanning unit to generate a three-dimensional volume image and determine whether the three-dimensional volume image is normal, a database configured to store the three-dimensional volume image generated by the processing unit, and an alarm sound output unit configured to, when data determined by the processing unit is not normal, provide notice thereof using any one or more of an alarm sound, an alarm light, and an alarm message.

### Advantageous Effects

Accordingly, the scan image discernment system in the three-dimensional ultrasound diagnostic apparatus of the present invention can enable a sonographer handling an ultrasonic wave to easily perform the scanning by determining whether an image is shaken or has a hidden part through a blurring determination algorithm and an image check algorithm.

In addition, the present invention can easily correct the scanned image by using a blurring determination algorithm and a hiding determination algorithm of a diagnostic apparatus and a three-axis acceleration sensor of a three-dimensional ultrasonic probing unit.

### DESCRIPTION OF DRAWINGS

FIG. 1 is a flowchart for showing a method of determining a scan image in a three-dimensional ultrasound diagnostic apparatus according to the present invention.

FIG. 2 is a block diagram showing a three-dimensional ultrasound diagnostic apparatus according to the present invention.

FIG. 3 shows a normal three-dimensional volume image according to the present invention.

FIG. 4 shows a blurred three-dimensional volume image according to the present invention.

FIG. 5 shows a hidden three-dimensional volume image according to the present invention.

### MODES OF THE INVENTION

The terms or words used in the specification and claims should not be construed as being limited to typical or dictionary meanings, but construed as the meaning and concept corresponding to the technical idea of the present invention on the basis of the principle that an inventor can appropriately define the concept of the term for describing his or her invention in the best method.

Accordingly, the configurations illustrated in embodiments and drawings described in the specification do not represent the technical idea of the present invention but are just exemplary embodiments. Thus, it should be understood that various equivalents and modifications may exist which can be replaced at a time when this specification is applied.

Hereinafter, exemplary embodiments of the present invention will be described in detail with reference to the accompanying drawings.

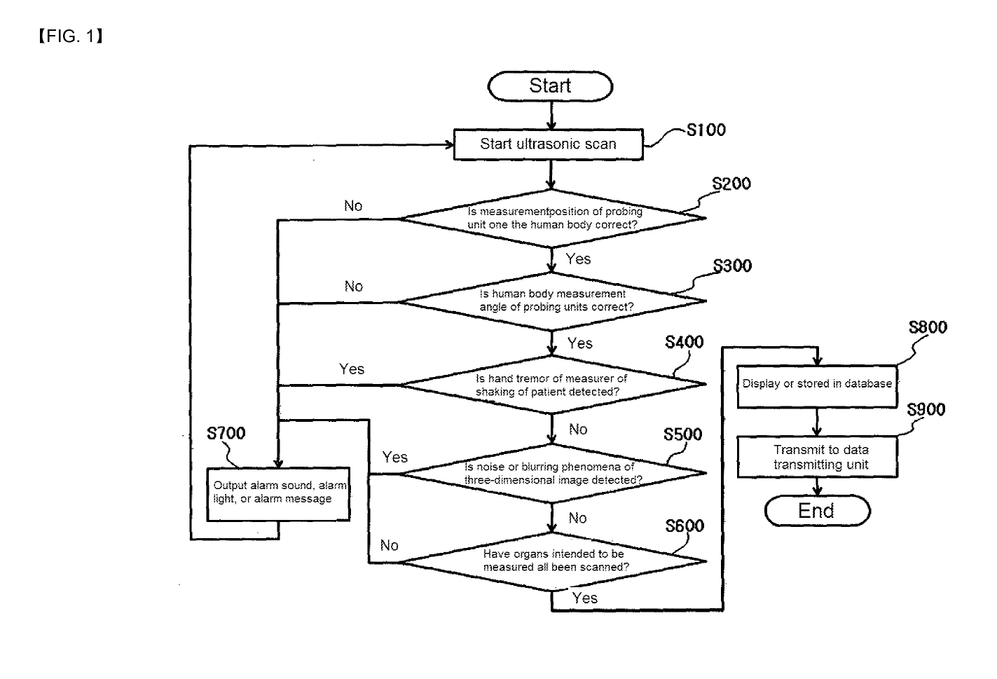

FIG. 1 is a flowchart for showing a method of determining a scan image in a three-dimensional ultrasound diagnostic apparatus according to the present invention. As shown in FIG. 1, a method of scanning an organ of a human body using a three-dimensional ultrasound diagnostic apparatus, determining whether a volume image of the scanned organ is shaken while the scanning is performed or whether the scanned organ is partially hidden due to a wrong scan direction, and providing notice thereof using any one or more of an alarm sound, an alarm light, and an alarm message to a sonographer such that the sonographer performs rephotographing will be described.

When a sonographer starts an ultrasonic scan (S100), it is determined whether a measurement position of a probing unit is correct on a human body using position data that is measured by a three-axis acceleration sensor or a 3-point space position sensor that is installed in the probing unit (S200). When the measurement position is not correct, any one or more of an alarm sound, an alarm light, and an alarm message is provided by an alarm sound output unit 400. When the alarm sound output unit 400 provides the alarm sound, the sonographer scans a corresponding part again.

When the position of the probing unit is correct, a measurement angle of the probing unit is determined by comparing a tip of the probing unit with a volume image value of an edge of an organ intended to be measured (S300). When a human organ is scanned, a portion or whole of the organ intended to be measured is hidden, and thus not shown according to the measurement angle of the probing unit. Thus, the step of determining the measurement angle of the probing unit is needed. That is, when the organ intended to be measured is not wholly shown due to a small measurement angle of the probing unit or is shown to overlap another organ due to a large measurement angle, the alarm sound output unit 400 provides notice thereof using any one or more of an alarm sound, an alarm light, and an alarm message.

When both of the position and angle of the probing unit are correct, it is determined whether a hand holding the probing unit is tremorous and whether a patient moves (S400). The tremor of the hand of the sonographer holding the probing unit is determined by installing a three-axis acceleration sensor or gyro sensor in the probing unit, determining a sensing value measured through the installed sensor, determining that the hand is tremorous when the sensing value is equal to or greater than a certain value, and outputting any one or more of an alarm sound, an alarm light, and an alarm message from the alarm sound output unit 400. The shaking of the patient is determined by installing any one or more of a three-axis acceleration sensor and a gyro sensor in a patient examination bed, determining a sensing value measured through the installed sensor, determining that the patient is shaken when the sensing value is equal to or greater than a certain value, and outputting at least one of an alarm sound, an alarm light, or an alarm message from the alarm sound output unit 400.

When the hand holding the probing unit is not tremorous and the patient does not move, noise or blurring detection is performed on the scanned three-dimensional volume image (S500). Examples of a filter used in an ultrasound diagnostic apparatus to remove the noise from the volume image include a Lee filter, an averaging filter, a frost filter, an enhanced frost filter, etc. The method of determining the blurring of the three-dimensional volume image includes detecting the blurring by determining a quality and a contrast of the image using a blurring algorithm. When the noise and blurring of the image are detected, the alarm sound output unit 400 provides notice thereof using any one or more of an alarm sound, an alarm light, and an alarm message.

When the noise or blurring of the scanned image is not detected, it is determined whether organs intended to be measured have all been scanned (S600). In this step, when the sonographer desires to scan a specific organ, whether organs intended to be measured have all been scanned is determined by comparing the scanned three-dimensional volume image with a standard three-dimensional volume image of each organ that is stored in a database 300 of the three-dimensional ultrasound diagnostic apparatus to automatically determine whether their forms and shapes are similar to each other. When the standard volume image stored in the database 300 is different from the scanned three-dimensional volume image, the alarm sound output unit 400 provides notice thereof using any one or more of an alarm sound, an alarm light, and an alarm message. In contrast, when the images are the same, the scanned image is displayed or stored in the database (S800). Subsequently, the stored three-dimensional volume image is transmitted to a data transmitting unit 600 (S900). The data transmitting unit 600 sends the three-dimensional volume image to a large hospital capable of providing a diagnosis or a hospital having a doctor who can provide a diagnosis, through a wired or wireless communication or a picture archiving and communication system (PACS) communication or a compact disk (CD) containing the image.

The alarm sound output unit 400 is allowed to output at least one of the alarm sound, alarm light, or alarm message when an error occurs upon at least one of the determination of the measurement location, the determination of the measurement angle in the second step, the determination of the tremor of the probing unit in the third step, the determination of the noise or blurring phenomena of the three-dimensional volume image in the fourth step, and the determination of whether the organs intended to be scanned have all been scanned in the fifth step.

The processing unit 200 can change the order of the first step of determining a measurement position of a probing unit on a human body, the second step of determining a measurement angle of the probing unit, the third step of detecting a tremor of a hand holding the probing unit or shaking of the human body, the fourth step of detecting noise or blurring phenomena in a three-dimensional volume image, and the fifth step of determining whether organs intended to be measured have all been scanned.

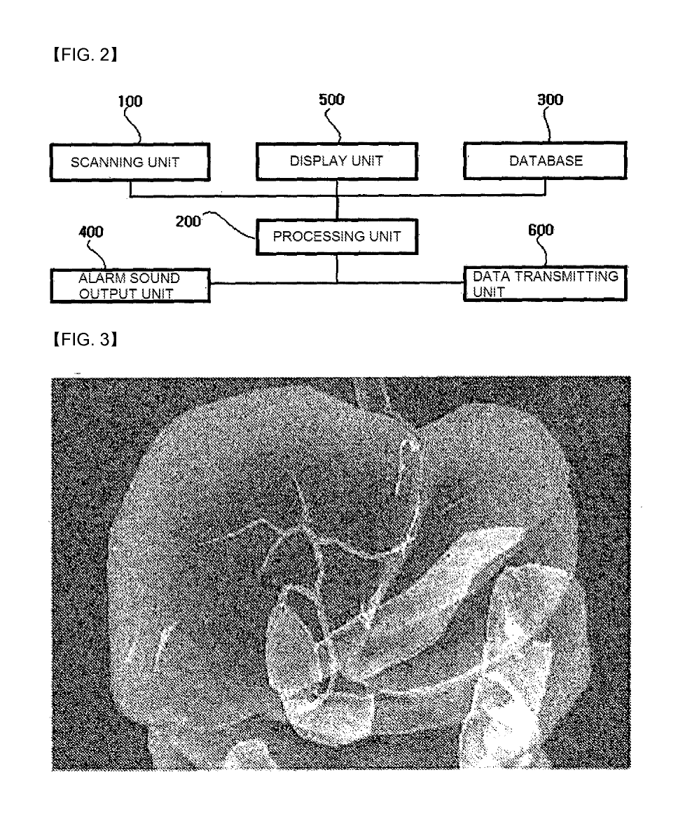

FIG. 2 is a block diagram showing a three-dimensional ultrasound diagnostic apparatus according to the present invention. As shown in FIG. 2, a scan image discernment device of the three-dimensional ultrasound diagnostic apparatus includes a scanning unit 100 configured to generate two-dimensional volume images of an inside of a human body using an ultrasonic signal, a processing unit 200 configured to combine the two-dimensional volume images acquired through the scanning unit 100 to generate a three-dimensional volume image and determine whether the three-dimensional volume image is normal, the database 300 configured to store the three-dimensional volume image generated by the processing unit, the alarm sound output unit 400 configured to, when the data determined by the processing unit is not normal, provide notice thereof using any one or more of an alarm sound, an alarm light, and an alarm message, a display unit 500 configured to display the three-dimensional data generated through the processing unit 200, and the data transmitting unit 600 configured to transmit the data generated by the processing unit 200 to a hospital for diagnosis through a wired/wireless communication, a CD, or a PACS system.

The scanning unit 100 includes a probing unit that scans an organ inside a human body using an ultrasonic signal to generate a volume image, and the probing unit includes any one or more of a three-axis acceleration sensor and a gyro sensor to determine a tremor of a hand holding the probing unit and transmits a sensing signal to the processing unit 200 when shaking is sensed.

The processing unit 200 includes an image determination algorithm and a blurring algorithm that are used to analyze the volume image scanned by the probing unit to find shaking, a hidden image, or a part hidden by another organ, which occur according to a position or angle of the probing unit. The image determination algorithm finds a blurred image using a ratio difference between a high frequency region and a low frequency region obtained through frequency analysis of the three-dimensional volume image.

The probing unit generates the two-dimensional volume image of an inside of the human body using an ultrasonic signal, senses a tremor of the probing unit using any one or more of the three-axis acceleration sensor and the gyro sensor, and transmits a movement sensing signal to the processing unit when the probing unit is tremorous.

In addition, the processing unit 200 determines whether the probing unit is correctly positioned by comparing a volume image value of an edge of the probing unit and a volume image value of an edge of an organ intended to be measured and determines a tremor of a hand holding the probing unit by building a three-axis acceleration sensor or gyro sensor in the probing unit and analyzing a sensing value. The shaking of the patient being given medial treatment is determined by installing a three-axis acceleration sensor or a gyro sensor in a patient examination bed and analyzing a sensing value from the installed sensor.

The processing unit 200 functions to determine a quality and a contrast of the three-dimensional volume image using a blurring algorithm to determine a blurring of the three-dimensional volume image, and functions to compare the scanned three-dimensional volume image with a standard three-dimensional volume image of each organ that is stored in the database 300 to determine whether their forms are similar to each other.

In addition, the processing unit 200 uses an image determination algorithm for finding shaking, a hidden image, or a part hidden by another organ, which occur according to a position or angle of the probing unit of the three-dimensional volume image, and the image determination algorithm determines the three-dimensional volume image using a ratio difference between a high frequency region and a low frequency region obtained through frequency analysis.

Furthermore, when there are a plurality of three-dimensional volume images for a scanned part, the processing unit 200 selects a clearest three-dimensional volume image, transmits the selected three-dimensional volume image to the display unit 500, and stores the selected three-dimensional volume image in the database 300.

The processing unit 200 compresses the stored three-dimensional volume image using an image compression algorithm and transmits the compressed three-dimensional volume image to the data transmitting unit 600. A representative example of the image compression algorithm is Wavelet. Wavelet conversion is appropriate for image processing because the conversion reflects a fact that, when a person sees an object, the person first looks out the entire outline of the object and then gradually comes to focus on its details. When typical JPEG image compression is performed at a high compression ratio, a block loss such as a block artifact occurs. However, the wavelet compression is performed on an entire image, and thus may basically solve a problem that occurs in JPEG image compression.

Various compression techniques may be used to compress the three-dimensional ultrasonic volume image in addition to the wavelet compression technique. The present invention uses the wavelet compression technique to choose which one of a contrast and a sharpness would be reduced and then perform compression.

The database 300 stores a three-dimensional volume image combined by the processing unit 200 and stores a standard three-dimensional volume image for each organ. The processing unit 200 compares the scanned three-dimensional volume image with the standard three-dimensional volume image stored in the database 300 and determines whether their forms and shapes are similar to each other.

When an erroneous volume image is detected by the processing unit 200, the alarm sound output unit 400 functions to provide notice thereof using any one or more of an alarm sound, an alarm light, and an alarm message, and the sonographer performs rephotographing upon recognizing the notice.

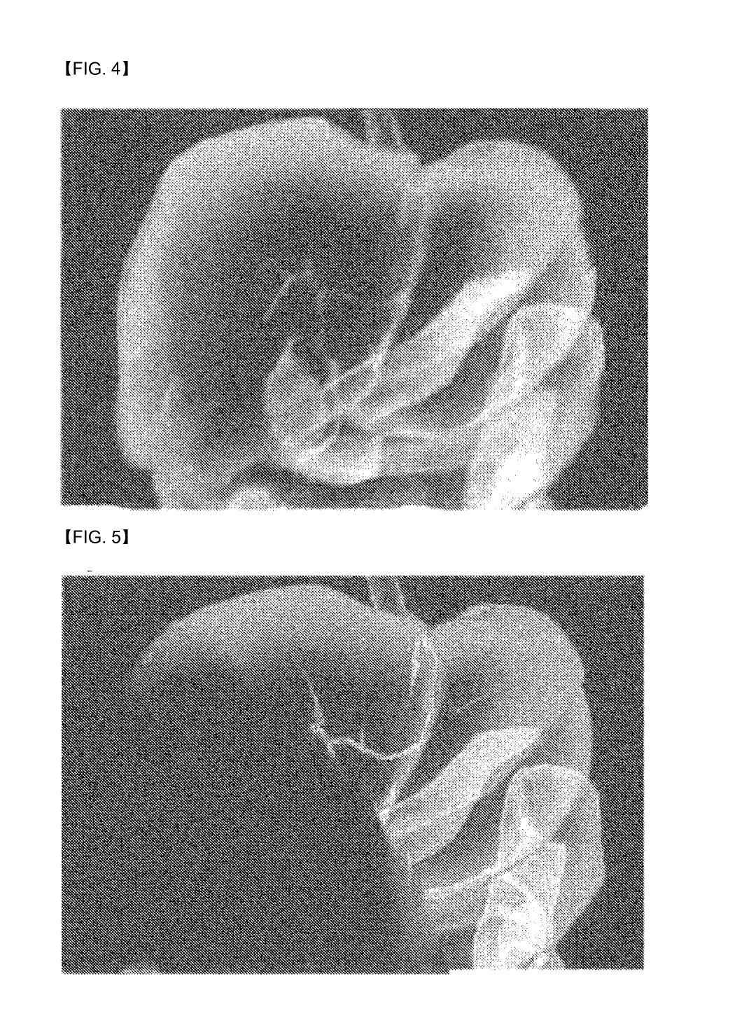

FIGS. 3 to 5 are associated with three-dimensional volume images that are scanned by the three-dimensional ultrasound diagnostic apparatus. FIG. 3 shows a normal three-dimensional volume image, FIG. 4 shows a blurred three-dimensional volume image, and FIG. 5 shows a three-dimensional volume image that is hidden by an organ or bone.

While the invention has been shown and described with reference to certain exemplary embodiments thereof, it will be understood by those skilled in the art that various changes in form and details may be made therein without departing from the spirit and scope of the invention as defined by the appended claims.

1. A method of determining a scan image in a three-dimensional ultrasound diagnostic apparatus, the method comprising
determining an image quality of a three-dimensional volume image before transmitting the three-dimensional volume image to a remote place.

2. The method of claim 1, further comprising:
a first step of determining, by a processing unit, a measurement position of a probing unit on a human body;
a second step of determining a measurement angle of the probing unit;
a third step of determining a tremor of a hand holding the probing unit or shaking of the human body;
a fourth step of determining noise or blurring phenomena in the three-dimensional volume image; and
a fifth step of determining whether organs intended to be measured have all been scanned.

3. The method of claim 2, wherein the first step comprises installing at least one of a three-axis acceleration sensor, a three-point spatial position sensor, and a gyro sensor and determining the measurement position of the probing unit on the human body using measured location data.

4. The method of claim 2, wherein the second step comprises comparing a volume image value of an edge of the probing unit with a volume image value of an edge of an organ intended to be measured, to perform the determination.

5. The method of claim 2, wherein the third step comprises determining the tremor of the hand using any one or more of a three-axis acceleration sensor or a gyro sensor included in the probing unit.

6. The method of claim 2, wherein the third step comprises installing any one or more of a three-axis acceleration sensor or a gyro sensor in a patient examination bed and determining the shaking of the human body through the sensor.

7. The method of claim 2, wherein the fourth step comprises determining the blurring of the three-dimensional volume image using a quality and a contrast of the three-dimensional volume image by performing a blurring algorithm on the three-dimensional volume image.

8. The method of claim 2, wherein the fifth step comprises comparing the scanned three-dimensional volume image with a standard three-dimensional volume image of each organ that is stored in a database of the three-dimensional ultrasound diagnostic apparatus to determine whether forms thereof are similar to each other.

9. The method of claim 2, wherein an alarm sound output unit outputs at least one of an alarm sound, an alarm light, or an alarm message when an error occurs upon at least one of the determination of the measurement location in the first step, the determination of the measurement angle in the second step, the determination of the tremor of the probing unit in the third step, the determination of the noise or blurring phenomena of the three-dimensional volume image in the fourth step, and the determination of whether the organs intended to be scanned have all been scanned in the fifth step.

10. The method of claim 2, wherein an order in which the first to fifth steps are performed is changeable.

11. An apparatus of determining a scan image of a three-dimensional ultrasound diagnostic apparatus, the apparatus comprising:
a scanning unit configured to generate two-dimensional volume images of an inside of a human body using an ultrasonic signal;
a processing unit configured to combine the two-dimensional volume images acquired through the scanning unit to generate a three-dimensional volume image and determine whether the three-dimensional volume image is normal;
a database configured to store the three-dimensional volume image generated by the processing unit; and
an alarm sound output unit configured to, when data determined by the processing unit is not normal, provide notice thereof using any one or more of an alarm sound, an alarm light, and an alarm message.

12. The apparatus of claim 11, further comprising:
a display unit configured to display the three-dimensional volume image generated through the processing unit; and
a data transmitting unit configured to transmit the three-dimensional volume image generated by the processing unit to a hospital for diagnosis through any one or more of a wired/wireless communication, a compact disk (CD), and a picture archiving and communication system (PACS) system.

13. The apparatus of claim 11, wherein the scanning unit comprises a probing unit, and the probing unit comprises any one or more of a three-axis acceleration sensor, a three-point spatial position sensor, and a gyro sensor.

14. The apparatus of claim 11, wherein the probing unit generates the two-dimensional volume images of the inside of the human body using the ultrasonic signal.

15. The apparatus of claim 13, wherein shaking of the probing unit is sensed using any one or more of the three-axis acceleration sensor and the gyro sensor of the probing unit, and a movement sensing signal is transmitted to the processing unit when the probing unit is shaken.

16. The apparatus of claim 11, wherein the processing unit determines the position of the probing unit by comparing a volume image value of an edge of the probing unit with a volume image value of an edge of an organ intended to be measured.

17. The apparatus of claim 11, wherein the processing unit determines a tremor of a hand holding the probing unit using a three-axis acceleration sensor or gyro sensor built in the probing unit.

18. The apparatus of claim 11, wherein the processing unit receives a sensing value generated when a patient moves through a three-axis acceleration sensor or gyro sensor installed in a patient examination bed and determines a tremor of the human body.

19. The apparatus of claim 11, wherein the processing unit determines blurring of the three-dimensional volume image by comparing a quality and a contrast of the three-dimensional volume image using a blurring algorithm.

20. The apparatus of claim 11, wherein the processing unit compares the scanned three-dimensional volume image with a standard three-dimensional volume image of each organ that is stored in the database to determine whether forms thereof are similar to each other.

21. The apparatus of claim 11, wherein the processing unit includes an image determination algorithm for analyzing the three-dimensional volume image to find shaking, a hidden image, or a part hidden by another organ that occurs according to a position or angle of the probing unit.

22. The apparatus of claim 21, wherein the image determination algorithm analyzes the three-dimensional volume image using a ratio difference between a high frequency region and a low frequency region through frequency analysis.

23. The apparatus of claim 11, wherein, when there are a plurality of three-dimensional volume images for a scanned part, the processing unit selects a clearest three-dimensional volume image and transmits the selected three-dimensional volume image to a display unit.

24. The apparatus of claim 11, wherein the processing unit compresses the three-dimensional volume image using an image compression algorithm and sends the compressed image to a data transmitting unit.

25. The apparatus of claim 24, wherein the image compression algorithm selects which one of a contrast and a sharpness is reduced using a wavelet algorithm before the compression.

26. The apparatus of claim 11, wherein the database stores the three-dimensional volume image combined by the processing unit and stores a standard three-dimensional volume image for each organ.

27. The apparatus of claim 11, wherein, when an incorrect image is detected by the processing unit, the alarm sound output unit provides the notice using any one or more of the alarm sound, the alarm light, and the alarm message.
